(19) United States
(12) Patent Application Publication
(10) Pub. No.: US 2012/0196285 A1
(43) Pub. Date: Aug. 2, 2012

(54) Methods for Enriching Microparticles or Nucleic Acids Using Binding Molecules

(75) Inventors: Patricia Okamoto, Shrewsbury, MA (US); Jan Godoski, Seattle, WA (US); Thomas Scholl, Westborough, MA (US)

(73) Assignee: Esoterix Genetic Laboratories, LLC, Burlington, NC (US)

(21) Appl. No.: 13/362,299

(22) Filed: Jan. 31, 2012

Related U.S. Application Data
(60) Provisional application No. 61/437,768, filed on Jan. 31, 2011.

(51) Int. Cl. C12Q 1/68

(52) U.S. Cl. 435/6.11; 435/6.12

(57) ABSTRACT

Methods for enriching specific microparticles, such as fetal microparticles or disease specific microparticles, in a biological sample are disclosed. In certain embodiments, the methods include combining a biological sample with a molecule that binds specific microparticles, and separating fractions of the biological sample, wherein the fraction that contains the binding molecule is enriched for the specific microparticles. Also disclosed are methods for enriching fetal nucleic acids by enriching fetal microparticles in a fraction of the biological sample and isolating nucleic acids from the enriched fraction. Methods for facilitating prenatal diagnosis of fetal chromosomal abnormalities are disclosed. In certain embodiments, the methods include combining a biological sample with a molecule that binds fetal microparticles, separating fractions of the biological sample, isolating nucleic acids from the fraction enriched for fetal microparticles, and analyzing the nucleic acids for the presence of a mutation.

### PRIOR RELATED APPLICATION

This application claims priority to U.S. provisional application No. 61/437,768, filed Jan. 31, 2011, the contents of which are hereby incorporated by reference in their entirety.

### FIELD

Embodiments of the present invention relate to methods for enriching a rare population of microparticles, cells, or nucleic acids from a complex mixture, such as blood, using specific binding molecules.

### BACKGROUND

Assessing and monitoring fetal health are of utmost importance during a pregnancy. Doctors and other medical professionals need to have the most accurate information available regarding the health of the fetus in order to minimize the risks to both the fetus and the mother during pregnancy and to optimize the number of healthy babies born. Understandably, expectant parents and relatives are also anxious for information about the health and condition of the fetus. It is desirable for this information to be available as early as possible so that the parents may make informed decisions regarding the pregnancy and any adverse medical conditions the fetus may have.

Access to fetal genetic material can provide significant information regarding the health of the fetus. For example, any genetic defects, such as chromosomal abnormalities, can be detected by analyzing fetal DNA. Chromosomal abnormalities include point substitutions, deletions, additions, translocations, or abnormal numbers of chromosomes or chromosome sets (aneuploidy). One example of aneuploidy is monosomy, a type of aneuploidy in which one chromosome of a pair is missing. Another type of aneuploidy is trisomy, in which there are three copies of the chromosome instead of a pair. Aneuploidy may be lethal or may cause one of several different genetic disorders, including Down syndrome (Trisomy 21), Edwards syndrome (Trisomy 18), Patau syndrome (Trisomy 13), and Turner syndrome (X instead of XX or XY).

For prenatal diagnosis of these conditions, the currently available procedures are limited and have certain disadvantages. One currently used procedure is amniocentesis, a medical procedure in which amniotic fluid containing fetal DNA is extracted from the amniotic sac where the fetus is developing, and then the fetal DNA is analyzed for any genetic abnormalities. Amniocentesis is usually performed between the fifteenth and twentieth week of the pregnancy (i.e., during the second trimester). Amniocentesis carries the risk of several significant complications, including preterm labor, fetal trauma, and even miscarriage of the fetus. Because the test cannot be performed reliably until the second trimester of the pregnancy, and because of the significant risks associated with the procedure, amniocentesis may not be a desirable procedure for many patients. Another procedure that is currently used is chorionic villus sampling (CVS), in which a sample of the placental tissue is taken and analyzed. CVS can be performed earlier than an amniocentesis (i.e., typically between 10-12 weeks of the pregnancy), but this procedure also carries increased risk of infection, fetal trauma, amniotic fluid leakage, and miscarriage. CVS is also subject to maternal cell contamination if maternal cells are not completely separated from the placenta. Therefore, because both amniocentesis and CVS are relatively invasive procedures and have certain health risks, these procedures may not be suitable for many patients.

Some fetal material is also present in the mother's bloodstream. This material includes fetal DNA contained in microparticles (also called vesicles, microvesicles, or apoptotic bodies) that are formed primarily when placental cells undergo apoptosis or other forms of cell death. Morphological changes occur during apoptosis or other forms of cell death, including a process known as “membrane blebbing,” which leads to the formation and release of these microparticles from the cell. Because these microparticles are formed from the cell membrane, the microparticles have on their surface biomarkers that are specific for the cell from which they formed. In addition, the contents of the microparticle can include nuclear material such as nucleic acids that are specific for the cell from which they were released. The sizes of the microparticles and the amount of microparticles present in the mother's bloodstream may vary based on the individual and, to a lesser extent, based on the gestational age of the fetus. In some instances, the amount of microparticles present may be correlated with adverse conditions during the pregnancy. Generally, the average size of the microparticles ranges from about 0.1 to about 1 μm. These microparticles are only present in the maternal bloodstream in very small amounts, and it is extremely difficult using known methods to distinguish the fetal DNA from the maternal DNA. If the fetal DNA could be isolated or purified, however, valuable information regarding the health of the fetus, including information about chromosomal or genetic abnormalities, could be obtained without imposing significant health risks to the mother or the fetus.

The isolation and enrichment of microparticles have other applications as well. For example, microparticles also are formed during the activation or apoptosis or other types of cell death of cancer cells, or the activation or apoptosis or other cell death of cells in certain other diseases. In addition, in patients that have cancer or certain other diseases, microparticles are released from the cells not only during cell death, but also intentionally by the cells, for example, during metastasis of the cancer. These disease specific microparticles may be found circulating in the patient's bloodstream or in other bodily fluids that come into contact with the disease or cancer cells.

Therefore, what is needed is a less invasive and reliable method for detecting fetal chromosomal or other genetic abnormalities of a fetus early in a pregnancy (i.e., during the first trimester). It is also desirable for such a method to be accurate and reproducible throughout the pregnancy (e.g., for monitoring the health of the fetus throughout pregnancy). Methods for enriching fetal microparticles and fetal DNA from maternal material are also needed. These methods are preferably efficient, informative, and inexpensive. What is also needed is a method to enrich disease specific microparticles (e.g., cancer microparticles) or the nucleic acids contained in such microparticles in order to detect, monitor, and analyze the diseases, tumors, or other cancers.

### SUMMARY

Methods for enriching a subpopulation of microparticles or nucleic acids in a biological sample are provided. In certain aspects, the enrichment methods include the steps of combining a biological sample with a binding molecule that binds a microparticle of the subpopulation, and separating two or more fractions of the biological sample, wherein the fraction that contains the binding molecule is enriched for the subpopulation of microparticles. In one embodiment, the subpopulation of microparticles are fetal microparticles. The biological sample may comprise, for example, at least one of a maternal whole blood sample, plasma sample, serum sample, or another blood fraction sample. In some embodiments, the binding molecule is an antibody or antibody fragment. Generally, the binding molecule specifically binds a fetal microparticle, but does not bind a maternal microparticle. In some embodiments, the two or more fractions are separated by flow cytometry, size exclusion filtration, or magnetic particle concentration. In certain embodiments, the biological sample is treated to remove endogenous antibodies prior to combining the biological sample with the binding molecule. In certain embodiments, the biological sample is combined with a binding molecule that binds maternal microparticles to remove maternal microparticles in the biological sample prior to combining the biological sample with the binding molecule.

In other aspects, methods for enriching fetal nucleic acids (e.g., DNA) in a biological sample are provided, including the steps of combining a biological sample with a binding molecule that binds a fetal microparticle, separating two or more fractions of the biological sample, wherein the fraction that contains the binding molecule is enriched for fetal microparticles, and isolating nucleic acids from the fraction that contains the binding molecule, thereby enriching fetal nucleic acids in the biological sample. The enriched fetal nucleic acids may be analyzed, for example, using digital PCR. The biological sample may comprise, for example, at least one of a maternal whole blood sample, plasma sample, serum sample, or another blood fraction sample. In some embodiments, the binding molecule is an antibody or antibody fragment. Generally, the binding molecule specifically binds a fetal microparticle, but does not bind a maternal microparticle. In some embodiments, the two or more fractions are separated by flow cytometry, size exclusion filtration, or magnetic particle concentration. In certain embodiments, the biological sample is treated to remove endogenous antibodies prior to combining the biological sample with the binding molecule. In certain embodiments, the biological sample is combined with a binding molecule that binds maternal microparticles to remove maternal microparticles in the biological sample prior to combining the biological sample with the binding molecule.

In certain aspects, less invasive methods for facilitating prenatal diagnosis of a chromosomal abnormality in a fetus are provided. The methods include the steps of obtaining a biological sample from a pregnant woman, combining the biological sample with a binding molecule that binds a fetal microparticle, separating two or more fractions of the biological sample, wherein the fraction that contains the binding molecule is enriched for fetal microparticles, isolating nucleic acids (e.g., DNA) from the fraction that contains the binding molecule, and analyzing the nucleic acids to detect the presence or absence of the chromosomal abnormality. The biological sample may comprise, for example, at least one of a maternal whole blood sample, plasma sample, serum sample, or other blood fraction sample. In certain embodiments, the chromosomal abnormality is an aneuploidy of chromosome 13, 18, 21, or X. In other embodiments, the chromosomal abnormality is a mutation associated with a disease. Alternatively, other genetic abnormalities may be detected. The fetal nucleic acids may be analyzed, for example, using digital PCR. In certain embodiments, the less invasive methods are reliable for samples obtained from a pregnant woman when the gestational age of the fetus is less than about 16 weeks. In some embodiments, the binding molecule is an antibody or antibody fragment. Generally, the binding molecule specifically binds a fetal microparticle, but does not bind a maternal microparticle. In some embodiments, the two or more fractions are separated by flow cytometry, size exclusion filtration, or magnetic particle concentration. In certain embodiments, the biological sample is treated to remove endogenous antibodies prior to combining the biological sample with the binding molecule. In certain embodiments, the biological sample is combined with a binding molecule that binds maternal microparticles to remove maternal microparticles in the biological sample prior to combining the biological sample with the binding molecule.

In other aspects, the disclosed methods also may be applied to the detection of a disease. For example, methods for facilitating diagnosis of cancer or another disease associated with cell activation, cell death, apoptosis, or release of microparticles (or combination thereof) are provided. The methods may include the steps of obtaining a biological sample from a patient, combining the biological sample with a binding molecule that binds a microparticle comprising a biomarker specific to the disease cells (e.g., cancer cells), separating two or more fractions of the biological sample, wherein the fraction that contains the binding molecule is enriched for disease specific microparticles, isolating DNA from the fraction that contains the binding molecule, and analyzing the DNA to detect the presence or absence of a mutation associated with the disease, wherein the presence of the mutation indicates that the patient has the disease. In certain embodiments, the disease is cancer. In some embodiments, the binding molecule is an antibody or antibody fragment. Generally, the binding molecule specifically binds a cancer-derived or disease-specific microparticle, but does not bind a normal cell-derived microparticle. In some embodiments, the two or more fractions are separated by flow cytometry, size exclusion filtration, or magnetic particle concentration. In certain embodiments, the biological sample is treated to remove endogenous antibodies prior to combining the biological sample with the binding molecule. In certain embodiments, the biological sample is combined with a binding molecule that binds microparticles formed from cells expected to be present in the sample to remove such microparticles in the biological sample prior to combining the biological sample with the cancer-specific or disease-specific binding molecule. The biological sample may comprise, for example, at least one of a whole blood sample, plasma sample, serum sample, other blood fraction sample, or sample of any bodily fluid that has come into contact with cancer or disease cells. The enriched nucleic acids may be analyzed, for example, using digital PCR. Also provided are methods for enriching microparticles comprising a disease specific biomarker in a biological sample and methods for enriching disease specific nucleic acids, by combining the biological sample with a binding molecule that binds the disease specific microparticle, separating two or more fractions of the biological sample, wherein the fraction that contains the binding molecule is enriched for disease specific microparticles.

### BRIEF DESCRIPTION OF THE FIGURES

Non-limiting embodiments of the methods of the invention are exemplified in the following figures.

FIG. 1 is a graph showing the genome equivalents of total DNA in microparticles captured with various antibodies as shown. The genome equivalents of total DNA were determined by digital PCR with primers to the β-globin gene. The left bar of each pair of bars reflects the genome equivalents isolated from a 32 week pregnant patient carrying a male fetus, and the right bar reflects the genome equivalents isolated from a non-pregnant female control. PLAP indicates that the results were obtained when an antibody that binds to placental alkaline phosphatase was used to capture the fetal microparticles. G233, G1, and G9 indicate that the results were obtained when each of those antibodies (which bind to different epitopes of human leukocyte antigen G (HLA-G)) was used to capture the fetal microparticles. CD41 indicates that the results were obtained when an antibody that binds CD41 (a marker for platelets) was used. For the CD41 results, the left bar of that pair of bars is not observable in this figure. Fas-L indicates that the results were obtained when an antibody that binds Fas-L (Fas ligand, a marker of apoptosis) was used.

FIG. 2 is a graph showing the genome equivalents of fetal DNA in microparticles that were captured with various biomarkers, as shown. The genome equivalents of fetal DNA were determined by digital PCR with primers to the Y chromosome-specific sequence Y49a (DYS1) gene. The left bar of each pair of bars reflects the genome equivalents isolated from a 32 week pregnant patient carrying a male fetus, and the right bar reflects the genome equivalents isolated from a non-pregnant female control. The biomarker used for microparticle capture in each experiment is shown below each pair of bars. Enrichment of fetal DNA was accomplished using the anti-PLAP antibody for microparticle capture for this sample. For the PLAP results, the right bar of that pair of bars is not observable in this figure. For the G233 results, the left bar of that pair of bars is not observable in this figure. For the G1 results, the left bar of that pair of bars is not observable in this figure. For the G9 results, the right bar of that pair of bars is not observable in this figure. For the CD41 results, the right bar of that pair of bars is not observable in this figure. For the Fas-L results, the right bar of that pair of bars is not observable in this figure.

FIG. 3 shows the percent yield of DNA after enrichment by microparticle capture and as determined by digital PCR. Panel A is a graph showing the percent yield of total DNA after enrichment, and Panel B is a graph showing the percent yield of fetal DNA after enrichment. The yield is the amount of total or fetal DNA relative to the amount present in the maternal plasma prior to microparticle capture (i.e., 1350 genomic equivalents (GE)/mL plasma and 194 GE/mL plasma for total and fetal DNA, respectively, before capture). The left bar of each pair of bars reflects the genome equivalents isolated from a 32 week pregnant patient carrying a male fetus, and the right bar reflects the genome equivalents isolated from a non-pregnant female control. The biomarker used for microparticle capture in each experiment is shown below each pair of bars.

FIG. 4 is a graph showing the enrichment of fetal DNA obtained after capture with various biomarkers. The fold enrichment was calculated as the percent fetal DNA after capture, divided by the percent fetal DNA in the maternal plasma prior to microparticle capture (e.g., 2-fold enrichment is a doubling of the fetal fraction; 1 fold is no enrichment). The plasma samples used in this experiment were from a 32 week pregnant patient carrying a male fetus. The biomarker used for microparticle capture in each experiment is shown below each bar.

### DETAILED DESCRIPTION

Embodiments of the present invention provide methods to enrich and quantify a rare population of microparticles, cells, or nucleic acids in a complex mixture. The methods involve the use of biomarkers for the capture of a specific population of microparticles or cells and thereby, enrichment of nucleic acids within these microparticles or cells. These methods also involve the quantification of these nucleic acids using sensitive methods known to one of skill in the art, such as single molecule counting methods as it is expected that the amount of nucleic acids isolated will be very low, highly pure, and may be below the detection limit for more conventional quantification methods such as spectrophotometry, dye intercalation, or quantitative PCR (qPCR) (although such conventional quantification methods may be appropriate in some instances). The disclosed enrichment methods have particular application for the isolation, enrichment, and detection of fetal DNA encapsulated in microparticles during apoptosis of placental cells. These fetal DNA-containing microparticles are known to be circulating in the maternal plasma throughout gestation. The disclosed enrichment methods also have application in the identification of mutations in rare disease cells (e.g., cancer cells) or disease specific microparticles (e.g., cancer microparticles) that are circulating in the blood.

### DEFINITION AND ABBREVIATIONS

The terms “microparticles,” “apoptotic bodies,” “microvesicles,” and “vesicles” are used interchangeably herein to refer to membrane-bound particles that may include genetic material and surface biomarkers from the cell from which they were derived, for example during apoptosis or other type of cell death. As used herein, the term “biomarker” refers to a molecule present on or in a particular cell type (e.g., a placental alkaline phosphatase protein on the surface of fetal cells). “Fetal microparticles,” “fetal derived microparticles,” “fetal-associated microparticles,” or the like are microparticles that may be found in the bloodstream or other biological sample of an expectant mother primarily due to the apoptosis of fetal cells. Fetal microparticles may have fetal-specific biomarkers on their surfaces and contain fetal DNA. “Disease microparticles,” “disease specific microparticles,” “disease-associated microparticles,” or the like are microparticles that have a biomarker that is specific to a particular disease. Cancer microparticles may have tumor or cancer specific markers on their surfaces. “Cancer microparticles,” “cancer cell derived microparticles,” “cancer-associated microparticles,” or the like are microparticles that may be found in the bloodstream or other bodily fluid of a patient with a cancer due to the apoptosis or other type of cell death of cancer cells, or other release from cancer cells. Cancer microparticles may have tumor or cancer specific markers on their surfaces.

As used herein, the term “biological sample” encompasses any sample obtained from a biological source suitable for use in the present methods in which a rare cell, microparticle, or nucleic acid is present in the same sample with other cells, microparticles, or nucleic acids. A biological sample can, by way of non-limiting example, include whole blood, serum, plasma, other blood fraction, amniotic fluid, cultured cells, and/or chorionic villi. In certain embodiments, the biological sample is a whole blood sample, plasma sample, serum sample, any other blood fraction sample, or a combination thereof. A biological sample may be obtained from an individual by any method known to one of skill in the art, and may be obtained directly (e.g., obtaining a blood sample by venipuncture from the individual) or indirectly (e.g., obtaining a biological sample from a healthcare provider, hospital, or practitioner that directly obtained the biological sample from the patient).

As used herein, the term “subject” is used to refer to a human or any non-human animal (e.g., mouse, rat, rabbit, dog, cat, cattle, swine, sheep, horse, or primate). Preferably, the subject is human. A subject can be a “patient,” which refers to a human presenting to a medical provider for diagnosis, treatment, or care for a condition or disease. The terms “patient” and “individual” may be used interchangeably herein. In one embodiment, the patient or individual is a woman and her condition is that she is pregnant. In some embodiments, a subject can be afflicted with or susceptible to a disease or disorder but may or may not display symptoms of the disease or disorder.

As used herein, the term “apoptosis” refers to a form of programmed cell death. Apoptosis causes morphological changes to the surface of a cell, often resulting in “blebbing” of the cell membrane, which causes microparticles to form. Because the microparticles are formed from the cell membrane, they carry any membrane-specific markers that the original cells also expressed (e.g., fetal-specific markers, disease-specific markers, or tumor-specific markers). In one example, apoptosis occurs naturally to placental or fetal cells during a pregnancy.

The term “enrichment” is used herein to refer to the concentration of a rare microparticle, cell, or nucleic acid from a complex mixture (e.g., the enrichment of a fetal microparticle in a maternal blood sample). The term “immuno-enrichment” also may be used to refer to enrichment methods in which an antibody, antibody fragment, or specific binding molecule is used to capture a rare microparticle, cell, or nucleic acid from a complex mixture. As used herein, the term “binding molecule” is used to refer to a molecule that specifically binds a rare particle, cell, or nucleic acid of interest in a complex mixture. In one embodiment, the binding molecule is an antibody, antibody fragment, protein receptor, or other protein that specifically binds the rare particle, cell, or nucleic acid of interest. In another embodiment, the binding molecule is a “biomarker,” which refers to a protein that specifically interacts with the rare particle, cell, or nucleic acid of interest. Enrichment is determined by comparing the ratio of the amount of target material (e.g., a fetal microparticle) to other material in the sample after capture has taken place, to the ratio of the target material to other material in the initial sample before capture. Enrichment results in an increase in the quality of the captured material with respect to detecting the target material (i.e., an increase in the ratio of target material to other material present).

The term “chromosomal abnormality” is used herein to refer to any kind of defect associated with a chromosome, including single or multiple base pair deletions, additions, and substitutions; translocations; or defects in the numbers of complete chromosomes or sets of chromosomes. The term “aneuploidy” refers to when one or more chromosomes are missing or are present in more than the normal number of copies. Aneuploidy is associated with many diseases or syndromes, including, but not limited to, Down syndrome, Edwards syndrome, Patau syndrome, and Turner syndrome.

“Polymerase chain reaction” or “PCR” refers to a molecular biology technique used to amplify (increase the concentration of) and/or quantify a small amount of nucleic acids (e.g., DNA). There are many forms of PCR, such as digital PCR or real time PCR, that are specialized for a particular purpose. For example, digital PCR is a refinement of the original PCR technique that is better able to provide absolute quantification of nucleic acids by partitioning individual nucleic acid molecules in separate regions. Various other PCR techniques, including those described herein (e.g., quantitative real time PCR, emulsion PCR, multiplex PCR, and digital PCR), are well known by those skilled in the art and may be used in the present methods depending upon the amount of nucleic acids present in a particular sample.

### Enrichment Methods for Fetal Microparticles in a Complex Composition

In some embodiments, the present invention provides methods for enriching fetal microparticles in a biological sample, by combining a biological sample with a binding molecule that binds a fetal microparticle, and separating two or more fractions of the biological sample, wherein the fraction that contains the binding molecule is enriched for fetal microparticles. In certain embodiments, the biological sample comprises at least one of a whole blood sample, plasma sample, serum sample, and other blood fraction sample. Because microparticles contain surface biomarkers and nucleic acids (e.g., DNA) from the cells that they were derived from, capture and enrichment of microparticles can be accomplished by using surface-specific biomarkers from the original cells. For example, for fetal DNA enrichment, binding molecules that bind to a fetal-specific protein, such as human leukocyte antigen G (HLA-G; histocompatibility antigen, class I, G), placental alkaline phosphatase (PLAP), or fetal fibronectin, or combinations thereof, may be used to identify and capture the fetal microparticles circulating in the maternal plasma. In other embodiments, the fetal specific protein is selected from the group consisting of placental lactogen, chromosome 21 open reading frame 105, adducin 1 (alpha), biotinidase, claudin 6, coagulation factor II (thrombin), coagulation factor VIII procoagulant component, major hisocompatibility complex class II DR beta 4, lactotransferrin, MAS1-oncogene, titin, vasohibin 1, chorionic somatomammotropin hormone 1, chorionic somatomammotropin hormone 2, chorionic somatomammotropin hormone-like 1, insulin-like growth factor binding protein 1, pregnancy specific beta-1 glycoprotein 1, H 19, tissue factor pathway inhibitor 2, pregnancy specific beta-1 glycoprotein 3, pregnancy specific beta-1 glycoprotein 9, pregnancy specific beta-1 glycoprotein 6, insulin-like growth factor 2, delta-like 1 homolog, proteoglycan 2, EF hand domain family member D1, pregnancy-specific beta-1-glycoprotein 7, a disintegrin and metalloproteinase domain 12, fibronectin 1, pappalysin 1, corticotropin releasing hormone, insulin-like growth factor binding protein 3, semaphorin 3B, collagen type IV alpha 1, pregnancy-specific beta-1-glycoprotein 5, pregnancy specific beta-1-glycoprotein 2, amiloride binding protein 1, S100 calcium binding protein P, growth differentiation factor 15, endothelial PAS domain protein 1, CD59 antigen, growth hormone 2, syndecan 1, serine protease inhibitor clade E member 2, collagen, type III, alpha 1, collagen type 4 alpha 2, phospholipase A2 group IIA, hydroxy-delta-5-steroid dehydrogenase 3 beta 1, Epstein-Barr virus induced gene 3, KiSS-1 metastasis-suppressor, KISS1-R (receptor), cytochrome P450 family 19 subA1, fibulin 1, keratin 18, polydom, transglutaminase 2, cyclin-dependent kinase inhibitor 1, adrenomedullin, protease serine 11, tissue inhibitor of metalloproteinase 2, follistatin-like 1, hydroxysteroid (17-beta) dehydrogenase 1, tissue inhibitor of metalloproteinase 3, epidermal growth factor receptor, glycoprotein nmb, chorionic gonadotropin beta polypeptide 7, chorionic gonadotropin beta polypeptide 8, chorionic gonadotropin beta polypeptide 2, disabled homolog 2, tumor-associated calcium signal transducer 2, FLJ14146, family with sequence similarity 46 member A, cytochrome p450 family 11 subf A polypeptide 1, hydroxysteroid (17-beta) dehydrogenase 2, serine protease inhibitor clade E member 1, collagen type 1 alpha 1, heat shock 22 kDa protein 8, mannosidase alpha class 1C member 1, glypican 3, placenta-specific 1, novel MAFF like protein, calpain 6, G antigen family Cl, Rho-related BTB domain containing 3, collagen type 6 alpha 1, tumor suppressor candidate 3, EGF-like domain multiple 6, tachykinin 3, tachykinin 3 Receptor, secreted phosphoprotein 1, RAS p21 protein activator, lectin galactoside binding soluble 14 (pp113), tensin-like SH2 domain containing 1, cysteine-rich motor neuron 1, fibrillin 2, matrix metalloproteinase 11, chorionic gonadotropin beta polypeptide, chorionic gonadotropin beta polypeptide 5, trombosponding type 1 domain 3, paternally expressed 10, biglycan, collagen type XV alpha 1, serine proteinase inhibitor clade B member 2, death-associated protein kinase 1, transcription factor AP-2 alpha, transgelin, placental growth factor, microfibrillar associated protein 5, pappalysin 2 (plac3), phospholipid transfer protein, pleckstrin homology-like domain, family A, member 2, keratin 8, protein kinase inhibitor beta, insulin receptor, discs large homolog 5, hydroxysteroid (11-beta) dehydrogenase 2, bone morphogenetic protein 1, T-box 3, pregnancy-specific beta-1-glycoprotein 11, glial cells missing homolog 1, alkaline phosphatase placental-like 2, angiotension II receptor type 1, cadherin 5 type 2, frizzled-related protein, insulin-like 4 (placenta), inhibin beta A (activin A), COBL-like 1, transforming growth factor beta receptor III, tissue factor pathway inhibitor, stimulated by retinoic acid gene 6 homolog, junctional adhesion molecule 2, dickkopf homolog 1, vestigial like 1, rho-related BTB domain containing 1, brain-specific protein, interleukin 1 receptor type 1, steroid sulphatase, serine proteinase inhibitor clade H member 1, G protein-coupled receptor 126, and lectin galactoside binding soluble 13. Alternatively, biomarkers specific to apoptosis, such as Annexin V or Fas ligand (FasL), or combinations thereof can be used. In addition, combinations of fetal specific markers and apoptotic markers may be used.

In certain embodiments, the binding molecule used is an antibody or antibody fragment. In other embodiments, the binding molecule is a receptor or other protein that specifically binds the desired biomarker. Preferably, the binding molecule may bind a fetal microparticle, but does not bind a maternal microparticle. In certain embodiments, the antibody or antibody fragment binds to PLAP, HLA-G, or Fas-L. Examples of antibodies that bind HLA-G include G1, G9, and G233.

In some embodiments, the antibodies, antibody fragments, or other binding molecules can have a detectable label for direct detection of the microparticles. Examples of a detectable label include a fluorescent dye, a radioactive tag, a colorimetic tag, and the like that are known to one of skill in the art. As used herein, the terms “label” and “tag” are used interchangeably to refer to a moiety attached to the binding molecule or a secondary antibody protein. Alternatively, the binding molecule/microparticle complexes are indirectly detected. In some embodiments, a secondary antibody that has a detectable label and that can bind the microparticle-specific antibody, antibody fragment, or other binding molecule can be used to indirectly detect the desired population of microparticles.

In certain embodiments, the desired population of microparticles is further enriched prior to or after immuno-enrichment with the antibody, antibody fragment, or other binding molecule. For example, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is selectively depleted of maternal microparticles by using binding molecules specific for a maternal biomarker. Detection and enrichment can then be achieved via immuno-enrichment in which solid supports are used to separate the antibody/microparticle and/or biomarker/microparticle complex from the rest of the plasma. Flow cytometry also can be used prior to immuno-enrichment to sort the labeled microparticles by size, shape, and fluorescent signal.

In certain embodiments, the antibody, antibody fragment, or biomarker is bound directly to a solid support prior to enrichment. Alternatively, the binding molecule/microparticle complex can be formed first and then bound to a solid support for isolation either directly or indirectly via a secondary antibody conjugated to the support. In addition, prior to formation of the antibody/microparticle and/or biomarker/microparticle complexes, endogenous antibodies in the sample may be removed by methods known to one of skill in the art, as the endogenous antibodies may bind nonspecifically to the solid support during immuno-enrichment, thereby decreasing the efficiency of the process.

In some embodiments, the solid support is a polystyrene bead or resin. One example of a method for immunoprecipitation using an antibody as the binding molecule, bound to a resin as the solid support, is the PIERCE DIRECT IP KIT (Thermo Scientific, Rockford, Ill.). In other embodiments, the solid support may be a column, plate, well, tube, or the like. Other solid supports include, but are not limited to, magnetic beads or resin, agarose beads or resin, and polyacrylamide/bis-acrylamide resins. The separation of the biological sample into two or more fractions may occur, for example, by subjecting the sample to flow cytometry, size exclusion filtration, or magnetic particle concentration.

The biological sample may comprise at least one of a whole blood sample, a plasma sample, a serum sample, or any other blood fraction sample, and the sample may be obtained from the patient by any method known to one of skill in the art. Various methods for separating a whole blood sample into two or more blood fraction samples are well known to one of skill in the art. In one embodiment, a whole blood sample is obtained by venipuncture from an individual and then centrifuged using low speed centrifugation in order to separate the plasma fraction from the rest of the blood fractions.

### Methods for Enrichment of Fetal Nucleic Acids

Methods for enriching fetal nucleic acids (e.g., DNA) in a biological sample are also provided, which include combining a biological sample with a binding molecule that binds a fetal microparticle, separating two or more fractions of the biological sample, wherein the fraction that contains the binding molecule is enriched for fetal microparticles, and isolating DNA from the fraction that contains the binding molecule, thereby enriching fetal DNA in the biological sample. The biological sample may be, for example, a whole blood sample, plasma sample, serum sample, or other blood fraction sample. The binding molecule binds a fetal microparticle and does not bind a maternal microparticle, and may be, for example, an antibody, antibody fragment, receptor, or other specific binding protein. In some embodiments, the binding molecule binds to placental alkaline phosphatase (PLAP), human leukocyte antigen G (HLA-G), or Fas ligand (Fas-L). Examples of antibodies that specifically bind to HLA-G are G1, G9, and G233. In other embodiments, the binding molecule binds one of the fetal specific proteins listed in the previous section.

In certain embodiments of the DNA enrichment methods, the binding molecule has a detectable label. For example, the binding molecule may have a fluorescent tag, radioactive label, or colorimetric label. The binding molecule may be attached to a solid support for enrichment either directly or indirectly via a secondary antibody conjugated to the support. In some embodiments, the solid support is a polystyrene bead or resin. In other embodiments, the solid support is a column, plate, well, tube, or the like. In other embodiments, the solid support may be a magnetic bead or resin, an agarose bead or resin, or a polyacrylamide/bis-acrylamide resin.

The separation of the biological sample into two or more fractions may occur by subjecting the sample to flow cytometry, size exclusion filtration, or magnetic particle concentration. In certain embodiments, the desired population of microparticles is further enriched prior to or after immuno-enrichment with the antibody, antibody fragment, or other binding molecule. For example, prior to formation of the antibody/microparticle and/or biomarker/microparticle complexes, endogenous antibodies in the sample may be removed by methods known to one of skill in the art, as the endogenous antibodies may bind nonspecifically to the solid support during immuno-enrichment, thereby decreasing the efficiency of the process. In addition, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is first selectively depleted of maternal microparticles by using binding molecules specific for a maternal biomarker. Detection and enrichment can then be achieved via immuno-enrichment in which solid supports are used to separate the antibody/microparticle and/or biomarker/microparticle complex from the rest of the plasma. Flow cytometry can also be used prior to immuno-enrichment to sort the labeled microparticles by size, shape, and fluorescent signal. In certain embodiments, the antibody, antibody fragment, or biomarker is bound directly to a solid support prior to enrichment.

The isolation of nucleic acids from the enriched microparticle fraction may occur by one or more methods that are well known to one of skill in the art. For example, the microparticles may be solubilized directly on a solid support using standard molecular biology techniques. Examples of such methods include the use of detergents or chaotropic salts in order to solubilize or disaggregate the microparticles. One example of a DNA extraction method is the method used with the QIAAMP Circulating Nucleic Acid kit (Qiagen). Alternatively, in some instances, the sample could be incubated with Proteinase K for 30 minutes at 56° C. while shaking at 400 rpm, followed by heat inactivation at 95° C. for 20 minutes, centrifugation at 5,000 g for 5 minutes, and removal of the supernatant from the debris for further analysis. Various modifications may also be suitable for extraction in some embodiments. In addition, other suitable methods for DNA extraction are well known to one of skill in the art.

Fetal nucleic acid quantities can be determined in each fraction by a sensitive method such as real-time PCR or digital PCR. The fetal nucleic acids may then also be examined for any genetic defects or chromosomal abnormalities. In some embodiments, multiplex PCR may be used (i.e., more than one fetal gene may be amplified simultaneously in a single PCR reaction). Alternatively, the fetal nucleic acids may be analyzed by sequencing methods known to one of skill in the art. Other methods by which the target molecules may be amplified include, but are not limited to whole genome amplification, strand displacement amplification, rolling circle amplification, ligase chain amplification, and multiple PCR methods including quantitative real time PCR, emulsion PCR, and digital PCR. The amplified targets may be detected with methods such as, but not limited to fluorescence such as a probe, dye, or nucleotide; chemiluminescence; radioactivity; capillary electrophoresis; microarrays; sequencing; mass spectrometry; and nanostring technology. The disclosed enrichment methods may be performed as early as the first trimester of the pregnancy, and may be repeated throughout the pregnancy to continue to monitor the health of the developing fetus.

### Less Invasive Methods for Prenatal Diagnosis of Fetal Health

Less invasive methods for facilitating prenatal diagnosis of a chromosomal abnormality in a fetus are provided, including obtaining a biological sample from a pregnant woman, combining the biological sample with a binding molecule that binds a fetal microparticle, separating two or more fractions of the biological sample, wherein the fraction that contains the binding molecule is enriched for fetal microparticles, isolating nucleic acids from the fraction that contains the binding molecule, and analyzing the isolated nucleic acids to detect the presence or absence of the chromosomal abnormality.

In one embodiment, the chromosomal abnormality is a mutation that is associated with a disease. In certain aspects, the chromosomal abnormality may be an aneuploidy of chromosome 13, 18, 21, or X. In certain other aspects, the chromosomal abnormality is a paternally controlled allele. In certain other aspects, the chromosomal abnormality is a point mutation. In some embodiments, the less invasive methods are reliable for samples obtained from a pregnant woman when the gestational age of the fetus is less than about 16 weeks. In one embodiment, the noninvasive methods are reliable for samples obtained from a pregnant woman during her first trimester of pregnancy.

In some embodiments, the binding molecule binds a fetal microparticle and does not bind a maternal microparticle, and may be, for example, an antibody, antibody fragment, receptor, or other specific binding protein. In some embodiments, the binding molecule binds to placental alkaline phosphatase (PLAP), human leukocyte antigen G (HLA-G), or Fas ligand (Fas-L). Examples of antibodies that specifically binds to HLA-G are G1, G9, and G233. Other binding molecules may be used that bind to one of the fetal specific binding proteins listed above.

In certain embodiments of the nucleic acid enrichment methods, the binding molecule has a detectable label. For example, the binding molecule may have a fluorescent tag, radioactive label, or colorimetric label. The binding molecule may be attached to a solid support for enrichment either directly or indirectly via a secondary antibody conjugated to the support. In some embodiments, the solid support is a polystyrene bead or resin. In other embodiments, the solid support is a column, plate, well, tube, or the like. In other embodiments, the solid support is a magnetic bead or resin, an agarose bead or resin, or a polyacrylamide/bis-acrylamide resin. The separation of the biological sample into two or more fractions may occur by subjecting the sample to flow cytometry, size exclusion filtration, or magnetic particle concentration. In certain embodiments, the desired population of microparticles is further enriched prior to or after immuno-enrichment with the antibody, antibody fragment, or other binding molecule. For example, prior to formation of the antibody/microparticle and/or biomarker/microparticle complexes, endogenous antibodies in the sample may be removed by methods known to one of skill in the art, as the endogenous antibodies may bind nonspecifically to the solid support during immuno-enrichment, thereby decreasing the efficiency of the process. In addition, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is first selectively depleted of maternal microparticles by using binding molecules specific for a maternal biomarker. Detection and enrichment can then be achieved via immuno-enrichment in which solid supports are used to separate the antibody/microparticle and/or biomarker/microparticle complex from the rest of the plasma. Flow cytometry can also be used prior to immuno-enrichment to sort the labeled microparticles by size, shape, and fluorescent signal. In certain embodiments, the antibody, antibody fragment, or biomarker is bound directly to a solid support prior to enrichment.

The nucleic acid isolation from the enriched microparticle fraction may occur by one or more methods that are well known to one of skill in the art. For example, the microparticles may be solubilized directly on a solid support using standard molecular biology techniques. Examples of such methods include the use of detergents or chaotropic salts in order to solubilize or disaggregate the microparticles. One example of a DNA extraction method is the method used with the QIAAMP Circulating Nucleic Acid kit (Qiagen). Alternatively, in some instances, the sample could be incubated with Proteinase K for 30 minutes at 56° C. while shaking at 400 rpm, followed by heat inactivation at 95° C. for 20 minutes, centrifugation at 5,000 g for 5 minutes, and removal of the supernatant from the debris for further analysis. Various modifications of these extraction methods may also be suitable for extraction in some embodiments. In addition, other suitable methods for nucleic acid extraction are well known to one of skill in the art.

Fetal nucleic acids quantities can be determined in each fraction by a sensitive method such as real-time PCR or digital PCR. The fetal nucleic acids may then also be examined for any genetic defects or chromosomal abnormalities. In some embodiments, multiplex PCR may be used (i.e., more than one fetal gene may be amplified simultaneously in a single PCR reaction). Alternatively, the fetal nucleic acids may be analyzed by sequencing methods known to one of skill in the art. Other methods by which the target molecules may be amplified include, but are not limited to whole genome amplification, strand displacement amplification, rolling circle amplification, ligase chain amplification, and multiple PCR methods including quantitative real time PCR, emulsion PCR, and digital PCR. The amplified targets may be detected with methods such as, but not limited to fluorescence such as a probe, dye, or nucleotide; chemiluminescence; radioactivity; capillary electrophoresis; microarrays; sequencing; mass spectrometry; and nanostring technology. The disclosed enrichment methods may be performed as early as the first trimester of the pregnancy, and may be repeated throughout the pregnancy to continue to monitor the health of the developing fetus.

### Methods for Enriching Disease Specific Microparticles

The disclosed methods also can be applied to the detection of microparticles specific to diseases. For example, methods for enriching cancer microparticles or other disease specific microparticles in a complex mixture are provided, as well as methods for facilitating diagnosis of cancer or other diseases associated with cell death and apoptosis. Both the enrichment and diagnosis methods include combining a biological sample with a binding molecule that binds a disease specific microparticle, and separating two or more fractions of the biological sample, wherein the fraction that contains the binding molecule is enriched for disease specific microparticles. For diagnosis of a disease such as cancer, nucleic acids are then isolated from the fraction that contains the binding molecule and analyzed to detect the presence or absence of a mutation associated with the disease such as cancer, wherein presence of the mutation indicates that the individual has the disease. The biological sample may be a blood sample, plasma sample, other blood fraction sample, or a sample of any bodily fluid that has come in contact with cancer or disease cells (e.g., bile, urine, mucus, cerebrospinal fluid, peritoneal fluid, lymphatic fluid, etc.). The binding molecule may be an antibody, antibody fragment, receptor, or other specific binding protein that binds a disease microparticle and does not bind a normal cell-derived microparticle.

In certain embodiments of these methods, the binding molecule has a detectable label, such as a fluorescent tag, radioactive label, or colorimetric label. The binding molecule may be attached to a solid support for enrichment either directly or indirectly via a secondary antibody conjugated to the support. In some embodiments, the solid support is a polystyrene bead or resin. In other embodiments, the solid support is a column, plate, well, tube, or the like. In other embodiments, the solid support is a magnetic bead or resin, an agarose bead or resin, or a polyacrylamide/bis-acrylamide resin. The separation of the biological sample into two or more fractions may occur by subjecting the sample to flow cytometry, size exclusion filtration, or magnetic particle concentration. In certain embodiments, the desired population of cancer microparticles is further enriched prior to or after immuno-enrichment with the antibody, antibody fragment, or other binding molecule. For example, prior to formation of the binding molecule/microparticle complexes, endogenous antibodies in the biological sample may be removed by methods known to one of skill in the art to eliminate or reduce nonspecific binding of the endogenous antibodies to the solid support during immuno-enrichment. In addition, counterstains such as DAPI, propidium iodide, Hoechst, or other another stain known to those of skill in the art that also binds to nucleic acids under specific cellular conditions can be used to further subfractionate and enrich for those microparticles that contain nucleic acids. In other embodiments, the biological sample is selectively depleted of microparticles produced by a cell type that would be expected in the particular biological sample, by using binding molecules specific for a biomarker present on those cells. Detection and enrichment can then be achieved via immuno-enrichment in which solid supports are used to separate the binding molecule/microparticle complex from the rest of the sample. Flow cytometry can also be used prior to immuno-enrichment to sort the labeled microparticles by size, shape, and fluorescent signal. In certain embodiments, the antibody, antibody fragment, or biomarker is bound directly to a solid support prior to enrichment.

The nucleic acids isolation from the enriched microparticle fraction may occur by one or more methods that are well known to one of skill in the art. Nucleic acid quantities can be determined in each fraction by a sensitive method such as real-time PCR or digital PCR. In some embodiments, multiplex PCR may be used (i.e., more than one gene may be amplified simultaneously in a single PCR reaction). Alternatively, the nucleic acids may be analyzed by sequencing methods known to one of skill in the art. Other methods by which the target molecules may be amplified include, but are not limited to whole genome amplification, strand displacement amplification, rolling circle amplification, ligase chain amplification, and multiple PCR methods including quantitative real time PCR, emulsion PCR, and digital PCR. The amplified targets may be detected with methods such as, but not limited to fluorescence such as a probe, dye, or nucleotide; chemiluminescence; radioactivity; capillary electrophoresis; microarrays; sequencing; mass spectrometry; and nanostring technology.

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

### EXAMPLES

The present invention may be better understood by reference to the following non-limiting examples.

### Example 1

### Enrichment of Fetal DNA in Maternal Blood Sample

A whole blood sample was obtained from a pregnant woman carrying a male fetus at 32 weeks gestation or from a control female woman that was not pregnant. The blood samples were centrifuged at 1600 g for 10 minutes at 22-23° C. to separate the plasma fraction. The plasma samples were then spun an additional 10 minutes at 3500 g and 22-23° C. to remove cellular debris and platelets. Polystyrene beads were cross-linked with antibodies made against the fetal biomarker HLA-G, PLAP, or FasL. The plasma samples were then incubated for several hours at 4° C. with the antibody cross-linked beads to capture the fetal microparticles. After capture, the microparticles were solubilized directly on the beads using standard molecular biology methods (e.g., using detergents or chaotropic salts), and the DNA was characterized and quantified by digital PCR using targets specific for fetal DNA and for total DNA.

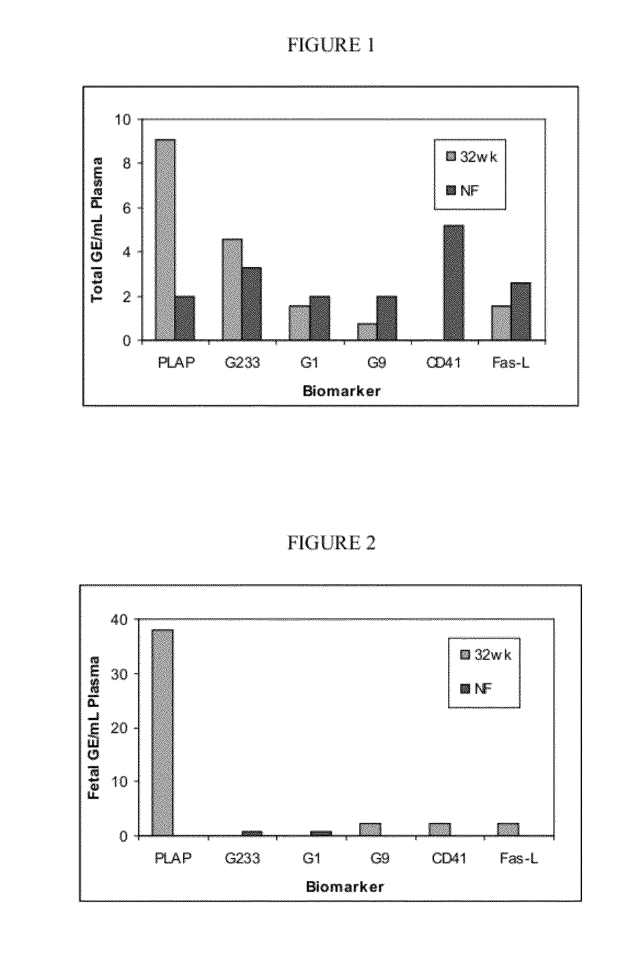

FIGS. 1 and 2 show the results of this digital PCR analysis. The genome equivalents of total DNA were determined by digital PCR with primers to the β-globin gene. The left bar of each pair of bars in FIG. 1 reflects the genome equivalents isolated from the 32 week pregnant patient carrying the male fetus, and the right bar reflects the genome equivalents isolated from the non-pregnant female control. PLAP indicates that the results were obtained when an antibody that binds to placental alkaline phosphatase was used to capture the fetal microparticles. G233, G1, and G9 indicate that the results were obtained when each of those antibodies (which bind to different epitopes of human leukocyte antigen G (HLA-G)) was used to capture the fetal microparticles. CD41 indicates that the results were obtained when an antibody that binds CD41 (a marker for platelets) was used. Fas-L indicates that the results were obtained when an antibody that binds Fas-L (Fas ligand, a marker of apoptosis) was used. FIG. 1 demonstrates that the antibody cross-linked beads are capable of capturing microparticles containing DNA.

FIG. 2 is a graph showing the genome equivalents of fetal DNA in the microparticles that were captured. The genome equivalents of fetal DNA were determined by digital PCR with primers to DYS1 gene because the fetus was male. Alternatively, specific sequences known to be contributed by the father also could be used for detection of fetal DNA. The left bar of each pair of bars in FIG. 2 reflects the genome equivalents isolated from the 32 week pregnant patient carrying a male fetus, and the right bar reflects the genome equivalents isolated from the non-pregnant female control. The biomarker used for microparticle capture in each experiment is shown below each pair of bars. Enrichment of fetal DNA was accomplished using the anti-PLAP antibody for microparticle capture for this sample. FIG. 2 demonstrates that antibody cross-linked beads are capable of capturing and enriching fetal microparticles containing DNA.

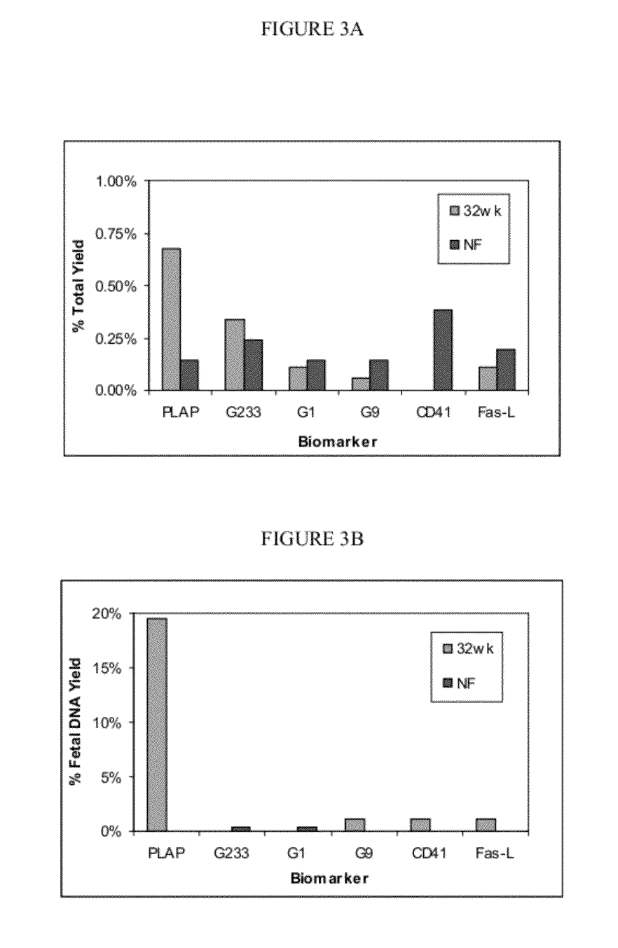

FIG. 3 shows the percent yield of DNA from this same experiment (after enrichment by microparticle capture and as determined by digital PCR). Panel A is a graph showing the percent yield of total DNA after enrichment, and Panel B is a graph showing the percent yield of fetal DNA after enrichment. The yield is the amount of total or fetal DNA relative to the amount present in the maternal plasma prior to microparticle capture (i.e., 1350 genomic equivalents (GE)/mL plasma and 194 GE/mL plasma for total and fetal DNA, respectively, before capture). The left bar of each pair of bars reflects the genome equivalents isolated from a 32 week pregnant patient carrying a male fetus, and the right bar reflects the genome equivalents isolated from a non-pregnant female control. The biomarker used for microparticle capture in each experiment is shown below each pair of bars, and anti-PLAP was used for enrichment. FIG. 3 further demonstrates that the antibody cross-linked beads are capable of capturing and enriching fetal microparticles containing DNA. Because PLAP is a later gestational age marker, use of this marker for capture and enrichment may be useful for a later gestational age screen. Other earlier gestational age markers will be useful for earlier prenatal diagnosis.

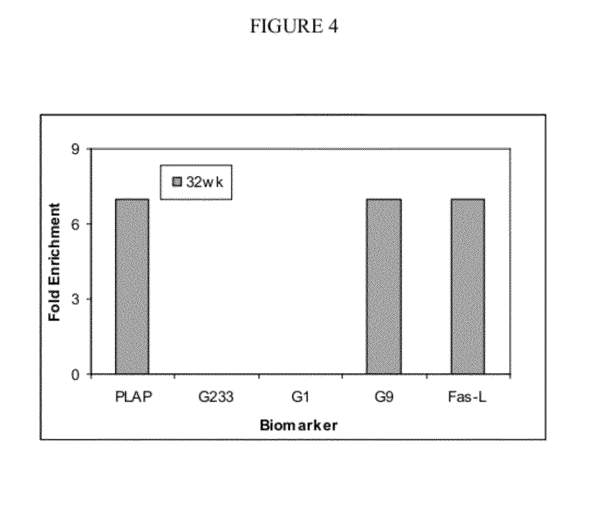

FIG. 4 is a graph showing the enrichment of fetal DNA obtained after capture with various biomarkers in this same experiment. The fold enrichment was calculated as the percent fetal DNA after enrichment, divided by the percent fetal DNA in the maternal plasma prior to microparticle capture (e.g., 2-fold enrichment is a doubling of the fetal fraction; 1 fold is no enrichment). The plasma samples used in this experiment were from a 32 week pregnant patient carrying a male fetus. The biomarker used for microparticle capture in each experiment is shown below each pair of bars. FIG. 4 further demonstrates that the antibody cross-linked beads are capable of successfully capturing and enriching fetal microparticles containing DNA.

### Example 2

### Prenatal Diagnosis of a Fetal Chromosomal Abnormality

A whole blood sample is obtained from a pregnant woman patient wishing to determine the chromosomal status of the fetus at 12 weeks gestation, and the sample is centrifuged at 1600 g for 10 minutes at 22-23° C. to separate the plasma fraction. The plasma fraction is then spun an additional 10 minutes at 3500 g and 22-23° C. to remove cellular debris and platelets. Polystyrene beads cross-linked to antibodies made against the fetal biomarker HLA-G are added to the plasma fraction and incubated for several hours at 4° C. to capture the fetal microparticles. After capture, the microparticles are solubilized directly on the beads using standard molecular biology methods. The DNA is characterized using standard molecular biology techniques to detect aneuploidy or other specific chromosomal abnormalities. No chromosomal abnormalities are detected, and this information is provided to the patient.

### Example 3

### Enrichment of Cancer Microparticles in Blood Sample

A whole blood sample is obtained from a patient suspected of having a lymphoma. The whole blood sample is centrifuged at 1600 g for 10 minutes at 22-23° C. to separate the plasma fraction, and then the plasma fraction is then spun an additional 10 minutes at 3500 g and 22-23° C. to remove cellular debris and platelets. Polystyrene beads cross-linked to antibodies made against a cancer cell biomarker are added to the plasma fraction and incubated for several hours at 4° C. to capture the cancer microparticles or cells circulating in the patient's blood. After capture, the microparticles are solubilized directly on the beads using standard molecular biology methods. The DNA is characterized using standard molecular biology techniques to detect a mutation associated with the lymphoma. The relevant mutation is detected, and this information is provided to the patient along with proposed treatment options.

While the invention has been described and illustrated with reference to certain embodiments thereof, those skilled in the art will appreciate that various changes, modifications and substitutions can be made therein without departing from the spirit and scope of the invention. All patents, published patent applications, and other non-patent references referred to herein are incorporated by reference in their entireties.

### REFERENCES

Gupta A K, Holzgreve, W, Huppertz, B, Malek, A, Schneider, H, Hahn, S (2004) Detection of Fetal DNA and RNA in Placenta-Derived Syncytiotrophoblast Microparticles Generated in Vitro. Clin Chem 50:2187-2190.
Piccin A, Murphy W G, Smith O P (2007) Circulating Microparticles: Pathophysiology and Clinical Implications. Blood Revs 21:157-171.
Orozco A F, Jorgez C J, Home C, Marquez-Do D A, Chapman M R, Rodgers J R, Bischoff F Z, Lewis D E (2008) Membrane Protected Apoptotic Trophoblast Microparticles Contain Nucleic Acids: Relevance to Preeclampsia. Am J Pathol 173:1595-1608.
Redman C W G and Sargent L L (2007) Microparticles and Immunomodulation in Pregnancy and Pre-eclampsia. J Biol Reprod 76:61-67.
Apps R, Gardner L, Moffatt A (2008) A Critical Look at HLA-G. Trends in Immunology 29: 313-324.
Huppertz B, Kadyrov M, Kingdom J C P (2006) Apoptosis and Its Role in the Trophoblast. AJOG 195:29-39.
Sykes P J, Neoh S H, Brisco M J, Hughes E, Condon J, Morley A A (1992). Quantitation of targets for PCR by use of limiting dilution. Biotechniques 13: 444-449
Vogelstein B, Kinzler K W (1999). Digital PCR Proc Natl Acad Sci USA. 96: 9236-9241.

That which is claimed is:

1. A method for enriching a subpopulation of microparticles in a biological sample, comprising
combining a biological sample with a binding molecule that binds a microparticle from the subpopulation, and
separating two or more fractions of the biological sample, wherein the fraction that contains the binding molecule is enriched for the subpopulation of microparticles.

2. The method of claim 1, wherein the biological sample comprises at least one of a whole blood sample, plasma sample, serum sample, and other blood fraction sample.

3. The method of claim 1, wherein the binding molecule is an antibody or antibody fragment.

4. The method of claim 3, wherein the antibody or antibody fragment has a detectable label.

5. The method of claim 3, wherein the antibody or antibody fragment is attached to a solid support.

6. The method of claim 1, wherein the two or more fractions are separated by flow cytometry, size exclusion filtration, or magnetic particle concentration.

7. The method of claim 1, wherein the biological sample is treated to remove endogenous antibodies prior to combining the biological sample with the binding molecule.

8. The method of claim 1, wherein the biological sample is combined with a binding molecule that binds maternal microparticles to remove maternal microparticles in the biological sample prior to combining the biological sample with the fetal specific binding molecule.

9. A method for enriching fetal nucleic acids in a biological sample, comprising
combining a biological sample with a binding molecule that binds a fetal microparticle,
separating two or more fractions of the biological sample, wherein the fraction that contains the binding molecule is enriched for fetal microparticles, and
isolating nucleic acids from the fraction that contains the binding molecule, thereby enriching fetal nucleic acids in the biological sample.

10. The method of claim 9, wherein the biological sample comprises at least one of a whole blood sample, plasma sample, serum sample, and other blood fraction samples.

11. The method of claim 9, wherein the binding molecule is an antibody or antibody fragment.

12. The method of claim 11, wherein the antibody or antibody fragment has a detectable label.

13. The method of claim 11, wherein the antibody or antibody fragment is attached to a solid support.

14. The method of claim 9, wherein the two or more fractions are separated by flow cytometry, size exclusion filtration, or magnetic particle concentration.

15. A method for facilitating prenatal diagnosis of a chromosomal abnormality in a fetus, comprising
obtaining a biological sample from a pregnant woman,
combining the biological sample with a binding molecule that binds a fetal microparticle,
separating two or more fractions of the biological sample, wherein the fraction that contains the binding molecule is enriched for fetal microparticles,
isolating nucleic acids from the fraction that contains the binding molecule, and
analyzing the nucleic acids to detect the presence or absence of the chromosomal abnormality.

16. The method of claim 15, wherein the chromosomal abnormality is an aneuploidy.

17. The method of claim 15, wherein the chromosomal abnormality is a mutation associated with a disease.

18. The method of claim 15, wherein the biological sample is obtained from the woman when the gestational age of the fetus is less than about 16 weeks.

19. The method of claim 15, wherein the biological sample is a whole blood sample or a plasma sample.

20. The method of claim 15, wherein the binding molecule is an antibody or antibody fragment.

21. The method of claim 20, wherein the antibody or antibody fragment has a detectable label.

22. The method of claim 20, wherein the antibody or antibody fragment is attached to a solid support.

23. The method of claim 15, wherein the two or more fractions are separated by flow cytometry, size exclusion filtration, or magnetic particle concentration.

24. A method for facilitating diagnosis of cancer, comprising
obtaining a biological sample from a patient,
combining the biological sample with a binding molecule that binds a cancer cell-derived microparticle,
separating two or more fractions of the biological sample, wherein the fraction that contains the binding molecule is enriched for cancer cell-derived microparticles,
isolating nucleic acids from the fraction that contains the binding molecule, and
analyzing the nucleic acids to detect the presence or absence of a mutation associated with cancer, wherein presence of the mutation indicates that the patient has cancer.

25. The method of claim 24, wherein the biological sample is combined with a binding molecule that binds microparticles formed from cells expected to be present in the biological sample to remove such microparticles in the biological sample prior to combining the biological sample with the cancer specific binding molecule

26. The method of claim 24, wherein the biological sample comprises at least one of a whole blood sample, plasma sample, serum sample, other blood fraction sample, or sample of any bodily fluid that has come into contact with cancer or disease cells.

27. The method of claim 24, wherein the binding molecule is an antibody or antibody fragment.

28. The method of claim 27, wherein the antibody or antibody fragment has a detectable label.

29. The method of claim 27, wherein the antibody or antibody fragment is attached to a solid support.

30. The method of claim 24, wherein the two or more fractions are separated by flow cytometry, size exclusion filtration, or magnetic particle concentration.
